(19) United States
(12) Patent Application Publication
(10) Pub. No.: US 2024/0285252 A1
(43) Pub. Date: Aug. 29, 2024

(54) MEDICAL SYSTEMS, DEVICES, AND RELATED METHODS FOR IMAGING, ASSESSING, AND/OR TREATING A PROSTATE

(71) Applicant: Boston Scientific Scimed, Inc., Maple Grove, MN (US)

(72) Inventors: Ajay GUPTA, Minneapolis, MN (US); Brenda Joy THEIS, Zimmerman, MN (US); Samuel Michael HANSON, Minneapolis, MN (US); Margaret GRILLEY, Saint Paul, MN (US); Thijs Andrew LARSON, Wayzata, MN (US); Zachary VAN FOSSAN, Eden Prairie, MN (US); Miah Noelle DUCKLOW, Woodbury, MN (US); Saipraneeth BAJJURI, Plymouth, MN (US); Thomas GOHRES, Chicago, IL (US); Timothy A. OSTROOT, Cokato, MN (US); Laura Emily RICHARDS, Plainville, MA (US); Stephanie HULANDER, Minneapolis, MN (US); Minhan JIANG, Minneapolis, MN (US); George Wilfred DUVAL, Sudbury, MA (US)

(73) Assignee: Boston Scientific Scimed, Inc., Maple Grove, MN (US)

(21) Appl. No.: 18/585,493

(22) Filed: Feb. 23, 2024

Related U.S. Application Data
(60) Provisional application No. 63/487,354, filed on Feb. 28, 2023.

(51) Int. Cl. A61B 8/12; A61B 1/06; A61B 8/08; A61B 17/34; A61B 18/00; A61B 18/14; A61M 25/00; A61M 25/01; A61M 25/06

(52) U.S. Cl. CPC A61B 8/12; A61B 8/08; A61B 8/485; A61B 18/1492; A61B 1/0676; A61B 2017/3413; A61B 2018/00547; A61B 2018/00613; A61M 25/0082; A61M 25/0108; A61M 25/065; A61M 2025/0681

(57) ABSTRACT

Medical systems are described, including a medical system that includes a sheath and a catheter. The sheath is configured to be positioned within a urethra of a patient. A proximal portion of the sheath includes a port configured to receive one or more fluids. The catheter is insertable into the sheath and configured to be movably positioned within the sheath in the urethra. A distal portion of the catheter includes one or more ultrasound transducers configured to perform ultrasound imaging of at least a portion of a prostate of the patient.

### CROSS-REFERENCE TO RELATED APPLICATIONS

This application claims the benefit of priority to U.S. Provisional Application No. 63/487,354, filed on Feb. 28, 2023, which is incorporated by reference herein in its entirety.

### TECHNICAL FIELD

Various aspects of this disclosure relate generally to systems, devices, and methods useful in medical procedures. More specifically, this disclosure relates to systems, devices, and methods for imaging, assessing, and/or treating tissue, for example, a prostate.

### BACKGROUND

Prostate cancer is a prevalent cancer affecting men globally. Prostate cancer imaging and biopsy are often challenging. For example, studies indicate that up to 30% of prostate cancers are isoechoic, such that the prostate cancers are not easily distinguishable from surrounding tissue via ultrasound imaging. Additionally, dense or irregular tumors may be difficult to target with biopsy needle(s), as the biopsy needle(s) may be deflected and/or yield a small or low amount of tissue. Existing biopsy devices seek to mitigate these drawbacks by using a spring-loaded needle and/or collecting tissue specimens either through a side fenestration or directly through the needle tip. Additionally, existing diagnostics or treatments may include magnetic resonance imaging (MRI) and/or transrectal ultrasound (TRUS). MRI techniques may provide better guidance for locating tumor(s), and TRUS techniques may provide real-time feedback on the placement of one or more needles targeting the tumor(s). However, MRI and TRUS techniques may be costly, require large or difficult to find capital equipment, and/or require multiple insertion or access locations in the patient.

The systems, devices, and methods of this disclosure may rectify some of the deficiencies described above, and/or address other aspects of the art.

### SUMMARY

Examples of this disclosure relate to, among other things, medical systems, devices, and methods. Each of the examples disclosed herein may include one or more of the features described in connection with any of the other disclosed examples.

In at least one example, a medical system may include a sheath and a catheter. The sheath may be configured to be positioned within a urethra of a patient. A proximal portion of the sheath may include a port configured to receive one or more fluids. The catheter may be insertable into the sheath and may be configured to be movably positioned within the sheath in the urethra. A distal portion of the catheter may include one or more ultrasound transducers configured to perform ultrasound imaging of at least a portion of a prostate of the patient.

The medical system may include one or more of the following aspects. The medical system may further include an insertion device with first and second lumens, and one of the first lumen or second lumen may be configured to receive the sheath or the catheter. The medical system may further include a needle. At least a portion of the needle may be configured to be movably positioned within the other of the first lumen or the second lumen of the insertion device. A distal portion of the needle may include a negative electrode portion and a positive electrode portion. The needle may be configured to be energized for irreversible electroporation therapy. The distal portion of the needle may include one or more echo markings. A distal portion of one or both of the first lumen or the second lumen may be sharpened or tapered. A proximal portion of the insertion device may include a female luer connection, and a proximal portion of the needle may include a male luer connection. The insertion device may further include a connection portion, an umbilicus, and an electronic connector to operably couple insertion device and the catheter to a console.

The needle may be an ultrasonic needle coupled to a piezoelectric transducer. The piezoelectric transducer may be configured to convey one or more of longitudinal movement, lateral movement, or rotational movement to the ultrasonic needle. The sheath may be flexible, and optionally a distal end of the sheath may be closed. The sheath may be translucent to ultrasound waves. The medical system may further include a needle configured to be delivered to the prostate of the patient transperineally. The needle may include at least one light source. The medical system may include one or more light sources positioned on the distal portion of the catheter. The distal portion of the catheter may include two transducers.

In another aspect, a medical system may include an ultrasound probe, an illumination device, and a needle. The ultrasound probe may be insertable into a rectum of a patient. The ultrasound probe may include a distal end comprising at least one transducer. The at least one transducer, when energized, may form an ultrasound field of view that at least partially overlaps with a prostate of the patient. The illumination device may be insertable into the rectum or a perineum of the patient. The illumination device may include at least one light source that, when energized, forms an illumination area that at least partially overlaps with the prostate of the patient. A distal end of the needle may be configured to biopsy and/or treat the prostate of the patient.

The medical system including an ultrasound probe, an illumination device, and a needle described above and elsewhere herein may include one or more of the following features. The illumination device may be coupled to the ultrasound probe. The illumination device may include two light sources coupled to a distal end of the ultrasound probe, and the two light sources may be adjacent to the transducer. The illumination device may include the needle. The illumination device may include two light sources coupled to a distal portion of the needle. The ultrasound probe may be configured to be delivered transrectally, and the needle may be configured to be delivered transperineally.

In yet another aspect, the present disclosure includes methods of treating a patient. The method may include inserting a sheath and a catheter through a urethra of the patient. The sheath may be flexible and may include a port in a proximal portion of the sheath. The sheath may include a closed distal end, and a distal portion of the catheter may include at least one ultrasound transducer. The method may also include delivering a fluid to the sheath through the port such that the fluid is between the catheter and the sheath. The method may further include imaging a prostate of the patient by activating or energizing the at least one ultrasound transducer of the catheter and longitudinally or rotatably moving the catheter. The method may also include removing the catheter and the sheath from the urethra.

The method may include one or more of the following features. Imaging the prostate may include obtaining multiple images by advancing the catheter distally, activating or energizing the one or more transducers multiple times, and longitudinally and/or rotatably moving the catheter to ultrasonically image different portions of the prostate. The method may further include applying pressure to the prostate transrectally and performing one or more elastography procedures.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” “generally,” and “approximately,” indicate a range of values within +/−10% of a stated value.

### BRIEF DESCRIPTION OF THE DRAWINGS

The accompanying drawings, which are incorporated in and constitute a part of this disclosure, illustrate various exemplary embodiments and together with the description, serve to explain the principles of the disclosure.

FIGS. 1A and 1B illustrate a medical system with portions of the medical system inserted into a patient, according to aspects of this disclosure.

FIGS. 2A and 2B illustrate additional views of the medical system of FIGS. 1A and 1B with portions of the medical system inserted into the patient distal portions, according to aspects of this disclosure.

FIG. 3 illustrates an exemplary process that may be performed with various portions of a medical system, according to aspects of this disclosure.

FIG. 4A illustrates various aspects of another medical system, and FIG. 4B illustrates portions of the medical system inserted into a patient, according to aspects of this disclosure.

FIGS. 5A-5C illustrate various aspects of yet another medical system, according to aspects of this disclosure.

FIGS. 6A-6C illustrate various aspects of an additional medical system, according to aspects of this disclosure.

FIG. 7 illustrates various aspects of another medical system, according to aspects of this disclosure.

FIGS. 8A and 8B illustrate various aspects of yet another medical system, according to aspects of this disclosure.

FIGS. 9A-9C illustrate various aspects of an additional medical system, according to aspects of this disclosure.

FIGS. 10-12 each illustrate an additional medical system for applying oscillatory movement to respective needles, according to aspects of this disclosure.

FIG. 13 illustrates various aspects of another medical system, according to aspects of this disclosure.

### DETAILED DESCRIPTION

Examples of this disclosure include systems, devices, and methods to facilitate and improve the efficacy, efficiency, and safety of medical procedures to image, assess, and/or treat one or more portions of a patient, for example, a prostate. For example, aspects of this disclosure may provide an operator (e.g., a physician, medical technician, or other medical service provider) with the ability to more easily assess, diagnose, or otherwise image (e.g., via ultrasonic imaging, photoacoustic imaging, etc.) a treatment site (e.g., a prostate). Aspects of this disclosure may alternatively or additionally provide the operator with the ability to more easily apply energy (e.g., irreversible electroporation, laser energy, etc.) from an energy source to the treatment site (e.g., the prostate). Furthermore, in some examples, aspects of this disclosure may provide the operator with the ability to more easily track the progress of various treatments. Aspects of this disclosure may allow an operator to deliver one or more medical devices to the treatment site in a sheathed or otherwise covered configuration, for example, such that the one or more medical devices may be reusable. Moreover, aspects of this disclosure may decrease the overall procedure time and cost, improve patient comfort, reduce the likelihood of infection, and/or otherwise improve visualization and/or patient safety. Additionally, aspects of this disclosure may be used in performing an ureteroscopic procedure.

Reference will now be made in detail to examples of this disclosure described above and illustrated in the accompanying drawings. Wherever possible, the same reference numbers will be used throughout the drawings to refer to the same or like parts.

The terms “proximal” and “distal” (e.g., arrow “P” and arrow “D” in various figures) are used herein to refer to the relative positions of the components of an exemplary medical device or an insertion device. When used herein, “proximal” refers to a position relatively closer to the exterior of the body or closer to an operator using the medical device or insertion device. In contrast, “distal” refers to a position relatively farther away from the operator using the medical device or insertion device, or closer to the interior of the body.

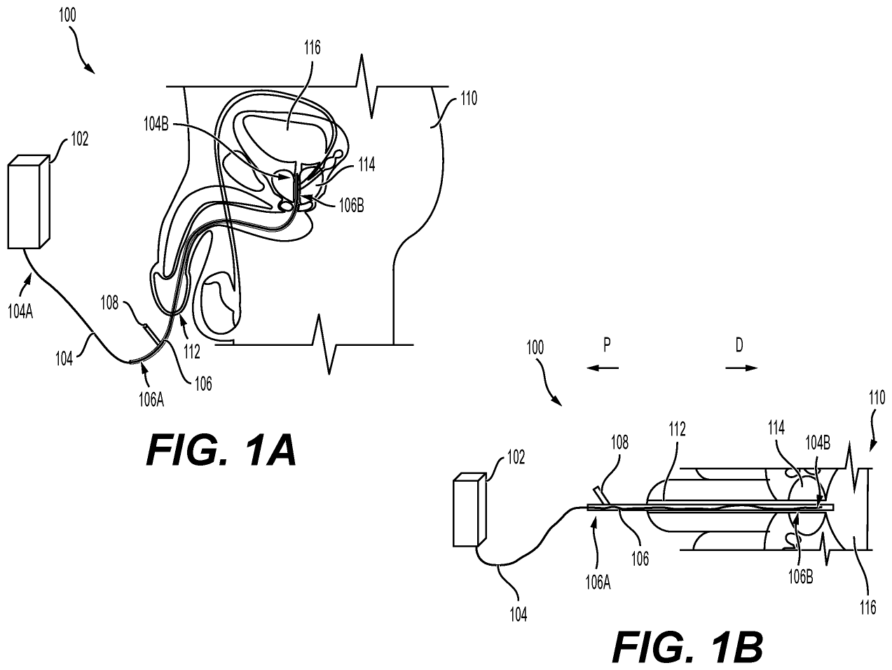

FIGS. 1A and 1B illustrate a medical system 100 that includes a console 102, an insertion device or catheter 104, and a sheath 106. As shown, a proximal end 104A of catheter 104 may be coupled to console 102, and a portion of catheter 104 (e.g., including a distal end 104B) may be positioned within a lumen of sheath 106. A proximal end 106A of sheath 106 may include a port 108 (e.g., extending at an angle relative to sheath 106). A distal end 106B of sheath 106 may be positioned within a portion of a patient 110 (e.g., transurethrally, transrectally, transperineally, etc.). For example, as shown, a portion of sheath 106 may be delivered through a urethra 112 of patient 110, such that distal end 104B of catheter 104 and distal end 106B of sheath 106 are positioned adjacent to a prostate 114 and/or a bladder 116 of patient 110. As discussed herein, catheter 104 may be an ultrasound catheter (e.g., an intravascular ultrasound (IVUS) imaging catheter), and sheath 106 may help to protect catheter 104. Additionally, catheter 104, along with console 102, may allow for visualization of one or more aspects of prostate 114, for example, direct 360° visualization of prostate 114. In some aspects, catheter 104, along with console 102, may include or otherwise be coupled to one or more visualization, assessment, and/or treatment techniques or combinations of visualization, assessment, and/or treatment techniques, for example, one or more visualization, assessment, and/or treatment techniques discussed herein.

Console 102 may include one or more controllers, energy or power sources, receivers, processors, etc., for example, configured to generate ultrasonic energy to be delivered to catheter 104 and/or to receive ultrasonic energy or other signals or information from catheter 104. For example, console 102 may be an IVUS console, for example, including one or more guidance systems (e.g., a multi-modality guidance system, although console 102 is not limited to these examples. Additionally or alternatively, console 102 may be configured to be coupled to one or more devices or systems, for example, for transurethral, transrectal, transperineal, or other treatment devices. Moreover, console 102 may include or otherwise be in communication with a user interface that may include a display (e.g., a touch screen display), for example, to receive user input(s) and/or to display images or other information. In some aspects, system 100 may include a catheter positioning and/or pull-back device, for example, such as a manual or automatic sled or other device to control a position of catheter 102 relative to patient 110.

As mentioned, catheter 104 includes proximal end 104A and distal end 104B. Proximal end 104A may be operably coupled or couplable (e.g., via a plug, connector, or other coupling mechanism) to console 102. Distal end 104B may include one or more transducers. Additionally, a portion, for example, a distal portion, of catheter 104 may be rigid or semi-rigid. In some aspects, catheter 104 may be a urethral scope. In other aspects, catheter 104 may be a different type of insertion device or scope (e.g., endoscope, duodenoscope, colonoscope, etc.). Catheter 104 may include a length configured to extend from console 102 to within patient 110, for example, within urethra 112 to be adjacent to prostate 114 and/or within a portion of bladder 116. In some aspects, catheter 104 may include a length of approximately 15 cm to approximately 40 cm, for example, approximately 21 cm. Moreover, catheter 104 may include a cross-sectional diameter or width (e.g., laterally relative to the longitudinal axis of catheter 104) of approximately 3 mm to approximately 10 mm. In some aspects, a cross-section of catheter 104 may include a tear-drop shape, for example, with a size of approximately 5 mm by approximately 7 mm.

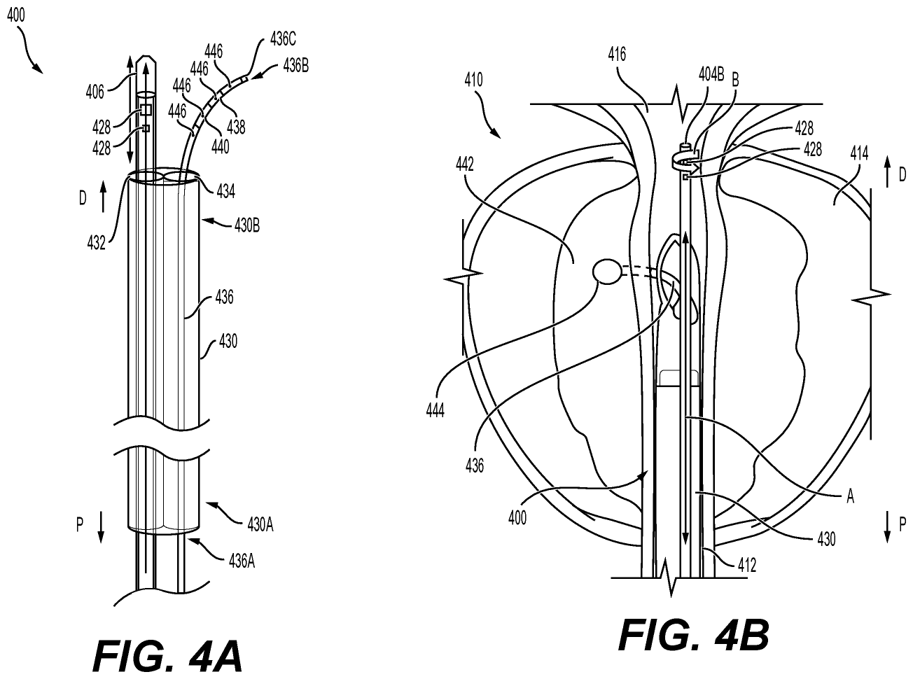

In these aspects, console 102 may provide signals to catheter 104 (e.g., to the one or more transducers at distal end 104B). For example, distal portion of catheter 104 with the one or more transducers 428 that may be positioned within urethra 112 (FIGS. 4A and 4B) may have penetration depths of approximately 25 mm to approximately 75 mm, for example, approximately 50 mm. The one or more transducers may be configured to generate ultrasonic energy in order obtain images during the positioning and/or repositioning of catheter 104. For example, the one or more transducers may obtain images during a pulling back (i.e., in a proximal direction) of catheter 104 (either alone or with sheath 106), and the positioning (e.g., distally and/or proximally) may be repeatable as many times as needed to obtain images of patient 110 (e.g., images of prostate 114). Alternatively or additionally, the one or more transducers may obtain images during a distal extension (in a distal direction) of catheter 104 (either alone or with sheath 106), and this positioning and movement may be repeatable as many times as needed to obtain images of patient 110 (e.g., images of prostate 114). In some aspects, the one or more transducers may obtain images during a pulling back and a distal extension of catheter 104 (either alone or with sheath 106).

Exemplary embodiments or approaches of this disclosure may employ a catheter that may be related to some extent to an IVUS catheter (mechanically scanned single element), i.e., the existing high frequency transducer element is replaced with a stack of low frequency (therapeutic) 1 MHz element with 30 MHz imaging overlaid. Alternatively, the catheter may include two transducers 428 (FIGS. 4A and 4B) side by side in close proximity to each other. Console 102 and/or catheter 104 may include one or more existing IVUS platforms including, for example, including one or more transducers that operate at various frequencies. For example, higher frequencies may have clearer resolution but smaller depth penetration (e.g., more attenuation), and lower frequencies may have lower resolution but larger depth penetration. In some aspects, the one or more transducers 428 (FIGS. 4A and 4B) may emit ultrasound waves at approximately 10 MHz to approximately 50 MHz, for example, approximately 15 MHz to approximately 40 MHZ.

In these aspects, catheter 104 may be more rigid and/or shorter than some existing IVUS catheters. As such, catheter 104 may be more robust, reduce the potential for electrical interference, reduce the potential for signal noise, and/or reduce the overall cost and/or time of production.

Sheath 106 may be a protective sheath, for example, to help insulate or isolate catheter 104 from bodily tissue and fluids (e.g., in urethra 112, bladder 116, or otherwise in patient 110). Sheath 106 may be or include a thin tube of biocompatible polymer material (e.g., including one or more layers of polymer material). For example, sheath 106 may be formed of a plastic material. In some aspects, sheath 106 may comprise or be formed of one or more metallic materials, for example, a combination of one or more metallic materials and one or more polymer materials. Suitable metallic materials include, for example, biocompatible metals and metal alloys. Suitable polymer materials include, for example, biocompatible polymers and copolymers, including elastomeric polymers. Proximal end 106A of sheath 106 may be open, for example, to receive a portion of catheter 104, and distal end 106B of sheath 106 may be closed, for example, to help insulate or isolate catheter 104 from patient 110. Moreover, as mentioned, sheath 106 may include port 108, which may receive a fluid (e.g., a saline fluid, water, ultrasonic gel, etc.) to be positioned around one or more portions of catheter 104 and be retained within sheath 106. In some aspects, proximal end 106A of sheath and/or port 108 may include one or more seals, luers, locks, etc. to help retain a portion of catheter 104 and/or the fluid within sheath 106.

Sheath 106 may be rigid or semi-rigid (e.g., at least partially flexible). For example, one or more portions of sheath 106 may be flexible, and one or more other portions of sheath 106 may be rigid. In other aspects, an entirety of sheath 106 may be flexible. For example, sheath 106 may be in the form of a thin condom-like device that may be slipped over or otherwise receive a portion of catheter 104. In further examples, sheath 106 may include a balloon, which may be inflated with a fluid (e.g., a liquid such as a saline solution, water, ultrasonic gel, etc., or a gas such as air), for example, to provide an area or a window for catheter 104 (e.g., including the transducer(s)).

Sheath 106 may be sized (e.g., with inflation) to oppose the urethral wall (e.g., including a radius of approximately 3 mm to approximately 10 mm, for example, approximately 5 mm to approximately 8 mm). Sheath 106 may help to provide an anchor for catheter 104, for example, exterior of patient 110), may help to retain the fluid (e.g., an imaging fluid), may help to allow for imaging without additional fluid in urethra 112, etc. In any of these aspects, one or more portions of sheath 106 is translucent to ultrasound waves. Sheath 106 may include a low attenuation. For example, in these aspects, sheath 106 does not include a braiding or a thickness that may impair the translucency to ultrasound waves or otherwise increase the attenuation in one or more portions along the length of sheath 106. Furthermore, in any of these aspects, sheath 106 may have a length of approximately 10 cm or longer, for example, from about 15 cm to about 50 cm, e.g., approximately 25 cm, approximately 35 cm, etc. Moreover, in some aspects, a portion (e.g., a distal portion) of catheter 104 and/or sheath 106 may be deflectable or otherwise steerable, for example, to help position and/or reposition catheter 104 and/or sheath 106.

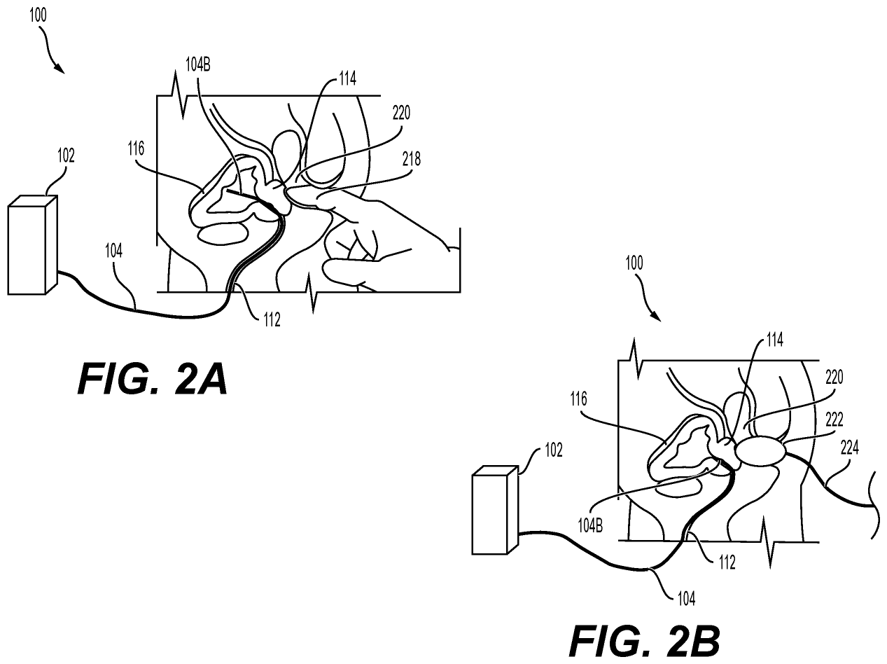

FIGS. 2A and 2B illustrate portions of system 100 and patient 110, along with different options to apply pressure on or adjacent to prostate 114, for example, to perform an elastography modality process or comparison. As discussed below, catheter 104 may be used to obtain one or more images in a first or natural configuration (e.g., as shown in FIGS. 1A and 1B) without externally applied pressure to prostate 114. Additionally, catheter 104 may be used to obtain one or more images in a second configuration (e.g., as shown in FIGS. 2A and 2B) with externally applied pressure to prostate 114. The two images (or the two sets of images) may be compared (e.g., by console 102) to help analyze or identify one or more aspects of tissue (e.g., of prostate 114), for example, such as a stiffness. The stiffness or one or more other aspects of the tissue may be indicative of one or more qualities of the tissue(s), for example, to help differentiate between healthy and unhealthy (e.g., cancerous or hyperplasic) tissues.

As shown in FIG. 2A, with catheter 104 and sheath 106 positioned within urethra 112 and adjacent to prostate 114 and/or bladder 116, a physician or other operator may apply pressure to prostate 114 directly. For example, the physician or a technician may insert a finger 218 into a rectum 220 of patient 110. The physician or technician may use finger 218 to apply pressure to prostate 114 digitally. As mentioned, system 100 may obtain one or more images in the digitally pressured configuration, and may compare the one or more images to one or more images in the natural or unpressured configuration.

Moreover, as shown in FIG. 2B, with catheter 104 and sheath 106 positioned within urethra 112 and adjacent to prostate 114 and/or bladder 116, a physician or other operator may apply pressure to prostate 114 with an inflatable balloon 222. For example, the physician or a technician may insert at least a portion of balloon 222 into rectum 220 of patient 110. The physician or technician may inflate balloon 222, for example, using an air tube 224 coupled to an air supply, to apply pressure to prostate 114. Balloon 222 may be inflated to a known or measurable pressure, for example, via a pressure gauge on or within balloon 222 or air tube 224. The pressure within balloon 222 may be adjustable. Additionally, balloon 222 may at least partially conform to the size and/or shape of rectum 220, for example, to apply pressure to prostate 114. As mentioned, system 100 may obtain one or more images in the balloon-pressured configuration, and may compare the one or more images to one or more images in the natural or unpressured configuration. In these aspects, system 100 may include one or more of balloon 222, air tube 224, and/or a pressure gauge.

In either situation (e.g., FIG. 2A or FIG. 2B), console 102 may compare various images to identify one or more properties of tissue, for example, one or more properties of the tissue of prostate 114. In these aspects, console 102 may perform an elastography modality process when comparing the images. In some aspects, power Doppler imaging may be used (e.g., with or without contrast) to help identify regions of increased blood flow. The power Doppler imaging may detect blood flow in the radial direction (e.g., relative to a longitudinal axis of catheter 104) with transurethral ultrasound waves passing in and out of prostate 114. Regions of increased blood flow, for example, in the tissue of prostate 114, may be indicative of unhealthy and/or cancerous tissue or a tumor.

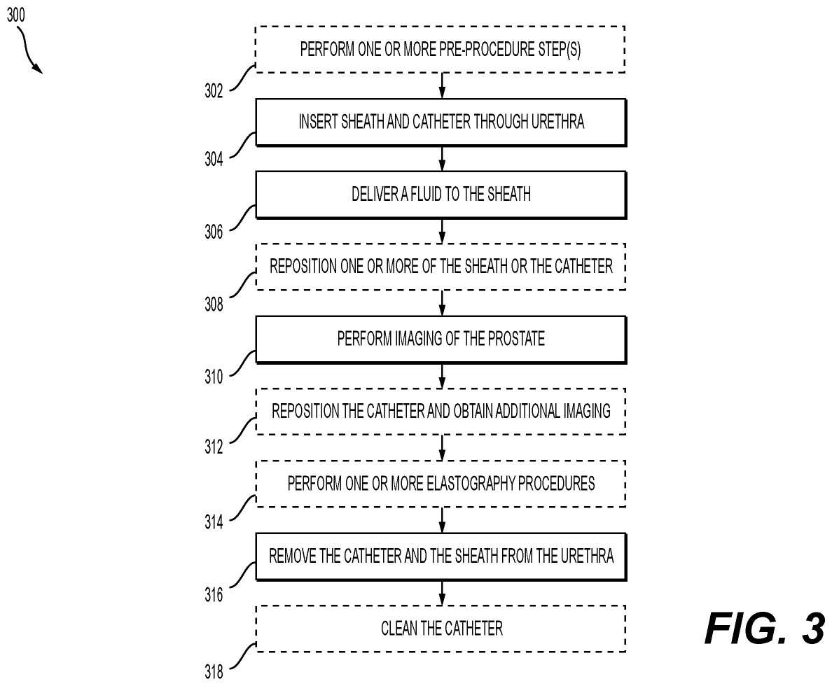

FIG. 3 is a flow diagram portraying an exemplary tissue analysis and/or diagnostic method 300, for example, at least partially using one or more components of system 100. Additionally, various aspects of method 300 may be performed using one or more aspects of the various systems discussed below. Although method 300 and various aspects discussed herein are directed to analysis, diagnosis, and/or other treatments of the prostate, it is noted that various aspects of this disclosure may be used to analyze, diagnose, or otherwise treat other organs or tissues. Method 300 may include performing one or more optional pre-procedure step(s) 302. Optional pre-procedure step(s) 302 may include patient 110 voiding bladder 116. Additionally or alternatively, optional pre-procedure step(s) 302 may include positioning patient 110 in on a procedure table, for example, in Trendelenburg's position (e.g., with the patient in a supine position on the procedure table and raising the bed such that the patient's feet are elevated relative to the patient's head). In some aspects, optional pre-procedure step(s) 302 may include applying anesthesia (e.g., local anesthesia) to urethra 112 (e.g., the urethral opening). Moreover, optional pre-procedure step(s) 302 may include applying a gel or lubricant to an outside of sheath 106.

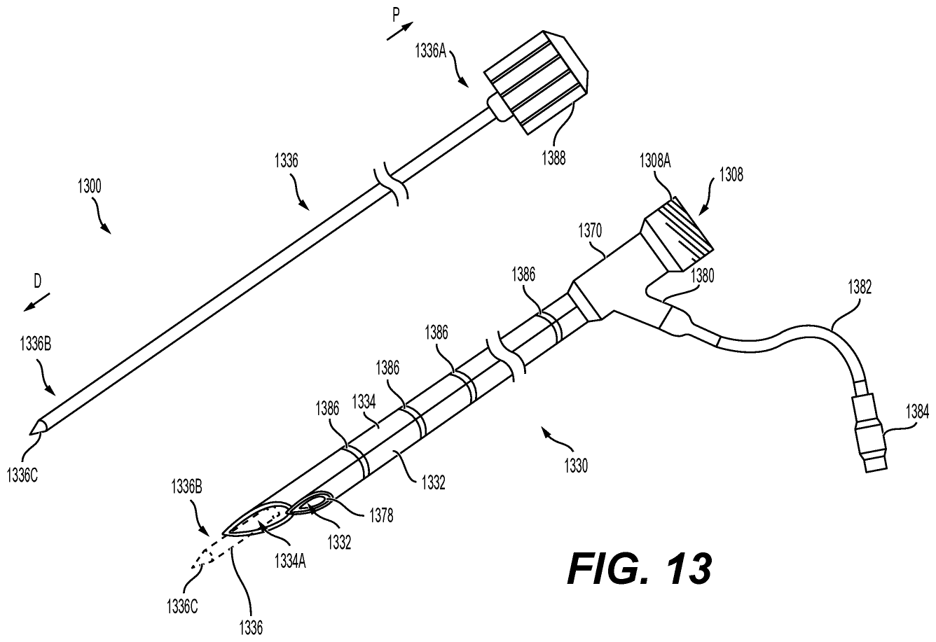

Next, method 300 includes a step 304, in which sheath 106 is inserted through urethra 112, for example, to a position adjacent prostate 114 and/or within a portion of bladder 116. In some aspects, step 304 includes positioning a portion of catheter 104 within sheath 106 before inserting sheath 106 through urethra 112. As mentioned, a proximal portion of sheath 106 may include one or more valves, luers, etc., for example, to form a seal around catheter 104. In some aspects, the position of distal end 104B of catheter 104 and/or distal end 106B of sheath may be determined or estimated, for example, based on a length of catheter 104 and/or sheath 106 inserted into urethra 112. In these aspects, catheter 104 and/or sheath 106 may include one or more measurement markings (e.g., FIG. 13), for example, indicative of an amount of catheter 104 and/or sheath 106 that has been inserted into patient 110. Moreover, in some aspects, step 304 may include using an introducer or dilator (e.g., a pushable introducer or dilator). For example, the introducer or dilator may be positioned within a portion of sheath 106 for insertion into urethra 112. Once sheath 106 is in place, the user may remove the introducer or dilator and insert catheter 104 within sheath 106.

Method 300 includes a step 306, in which a fluid (e.g., a saline solution, water, ultrasonic gel, etc.) may be delivered to sheath 106 to at least partially surround catheter 104. As mentioned above, the fluid may be delivered through port 108, and may be retained within sheath 106, for example, with one or more seals, luers, etc. on port 108.

Method 300 may also include an optional step 308, in which catheter 104 and/or sheath 106 are repositioned, e.g., based on images. For example, step 308 may include activating catheter 104 and obtaining one or more internal images. The operator may identify urethra 112, prostate 114, and/or bladder 116 via the one or more internal images, and may adjust the position of catheter 104 and sheath 106 based on the one or more internal images. In some aspects, a portion (e.g., a distal portion) of catheter 104 and/or sheath 106 may be deflectable or otherwise steerable, for example, to help position and/or reposition catheter 104 and/or sheath 106. Alternatively or additionally, step 308 may include one or more external imaging techniques to determine the position of catheter 104 within patient 110. For example, the operator may adjust the position of catheter 104 and sheath 106 within patient 110 based on the one or more images. In any of these aspects, distal end 104B of catheter 104 may be positioned just beyond the proximal end of prostate 114 and extending into a distal portion of bladder 116 (as shown in FIG. 1B).

Next, method 300 includes a step 310 in which imaging of prostate 114 is performed. For example, with distal end 104B of catheter 104 positioned distal to prostate 114, the one or more transducers may be activated. The operator may gradually pull-back or proximally retract catheter 104 (e.g., alone or with sheath 106). The operator may pull-back or proximally retract (or distally extend) catheter 104 over a distance of approximately 1 cm to approximately 5 cm, for example, approximately 2 cm to approximately 3 cm, such that distal end 104B of catheter 104 spans the length of prostate 114 in order to image various portions of prostate 114. In some aspects, step 310 may include rotating catheter 104, for example, such that the one or more transducers in distal end 104B may also be rotated to obtain the images of various portions of prostate 114. As mentioned above, the one or more transducers may allow for system 100 (e.g., console 102 and/or catheter 104) to obtain radial cross-sectional images of prostate 114. In some aspects, the radial cross-sectional images of prostate 114 may be stitched together or otherwise combined, for example, by software stored on console 102 or otherwise a part of system 100. In these aspects, the radial cross-sectional images may allow for the operator and/or system 100 to determine a volume or size of prostate 114 or portions of prostate 114, a volume or size of unhealthy tissue (e.g., cancerous tissue, a tumor, or hyperplasic tissue), etc. In some aspects, step 310 may include one or more other one or more imaging, visualization, assessment, and/or treatment techniques discussed herein. Furthermore, in some aspects, the imaging of step 310 may be used to track the size of prostate 114 or unhealthy tissue over time.

In an optional step 312, the operator may reposition catheter 104 (e.g., alone or with sheath 106) in order to obtain one or more additional images of prostate 114. For example, optional step 312 may include repositioning catheter 104 to a position distal to prostate 114 and repeating step 310.

Furthermore, in another optional step 314, the operator may perform one or more elastography procedures. As discussed above, the one or more elastography procedures may include obtaining a first image or first set of images (e.g., via the one or more transducers of catheter 104) with prostate 114 in a natural configuration, and also obtaining a second image or second set of images (e.g., via the one or more transducers of catheter 104) with pressure applied to prostate 114. As discussed with respect to FIGS. 2A and 2B, applying pressure to prostate 114 may include pressure applied digitally with finger 218 of an operator in rectum 220 or pressure applied by balloon 222 inflated in rectum 220 (e.g., via air tube 224). The one or more elastography procedures may include comparing the first image or first set of images with the second image or the second set of images, for example, to detect blood flow in and/or around prostate 114.

After satisfactory images have been obtained, method 300 includes removing catheter 104 and sheath 106 from urethra 112 of patient 110 in a step 316. Step 316 may include removing catheter 104 from sheath 106 and then removing sheath 106. Additionally, when sheath contains fluid, step 316 may include removing some or all of the fluid within sheath 106, for example, after removing catheter 104 from patient 110 and before removing sheath 106 from patient 110. Moreover, in some aspects, step 316 may include discarding catheter 104 (e.g., a portion of catheter 104 if a distal portion of catheter 104 is removable and/or replaceable) and/or sheath 106.

Furthermore, in some aspects, method 300 includes an optional step 318 that includes cleaning (e.g., wiping down, disinfecting, etc.) catheter 104 and/or sheath 106. Even though catheter 104 is surrounded by sheath 106 when within patient 110, catheter 104 may be cleaned before another use (e.g., with patient 110 or with another patient). Alternatively or additionally, sheath 106 may be cleaned before another use.

In these aspects, catheter 104 may be specialized or otherwise adapted for insertion through urethra 112. For example, catheter 104 may include a shorter length (e.g., between proximal end 104A and distal end 104B) than existing ultrasound imaging catheters (e.g., intravascular ultrasound catheters). Catheter 104 may also be stiffer than existing ultrasound imaging catheters.

Moreover, transurethral ultrasound may help to provide improved and/or clearer visualization of tissue (e.g., tissue of prostate 114) compared to other imaging techniques (e.g., as compared to transrectal ultrasound). In some aspects, system 100 may be less likely to receive interference from other components or systems in a diagnostic setting. In some aspects, system 100 and method 300 may be less dependent on operator skill or technique than other diagnostic systems, and may also help to allow for repeated procedures to monitor tissue (e.g., cancerous tissue or tissues otherwise suspected of disease in prostate 114) over time. As mentioned, system 100 and method 300 may help to visualize blood flow, for example, in a power Doppler mode. Visualizing blood flow may help to detect microvascular proliferation and/or detect flow of one or more injected fluids or materials (e.g., therapeutic agents such as TheraSphere™ radioactive glass microspheres), which may be injected with a microcatheter or needle. In some aspects, the injected fluids or materials are not radiopaque, and thus not readily detectable with radio frequency waves, but may be detectable with ultrasound waves. Additionally, one or more of catheter 104 and/or sheath 106 may be configured for single use, e.g., comprising inexpensive materials, being disposable, etc. Furthermore, system 100 and method 300 may be used without ionizing radiation. For example, method 300 may be performed without general anesthesia (e.g., in an office setting), with minimal or limited preparation time and/or procedure time compared to a transrectal ultrasound. In some aspects, system 100 and method 300 may include a reduced risk of infection compared to other diagnostic procedures (e.g., as compared to other medical procedures for imaging, assessment, and/or treatment of the prostate). Furthermore, in some aspects, the transurethral ultrasound of method 300 using system 100 may be more culturally acceptable and/or more comfortable for patient 110, for example, compared to a transrectal ultrasound and/or a digital rectal examination of prostate 114. In some aspects, system 100 and/or method 300 may help to identify aggressive cancers, guide therapy decisions, and/or develop a continuum of care, which may help to improve patient care.

Turning now to FIGS. 4A and 4B, another exemplary medical system 400 of this disclosure is illustrated. System 400 includes a catheter 404 and a sheath 406, which may include any of the features of catheter 104 and sheath 106 discussed above. Additionally, system 400 includes an insertion device 430, for example, a dual lumen insertion device with a first lumen 432 and a second lumen 434. Insertion device 430 includes a proximal end 430A, for example to be positioned external of a patient, and a distal end 430B, for example to be positioned internal to the patient (e.g., adjacent to one or more portions of a prostate 414). Furthermore, system 400 includes a needle 436 (e.g., an irreversible electroporation, or “IRE,” needle), including a proximal end 436A and a distal end 436B. As discussed in detail below, needle 436 may be a bipolar or unipolar (or monopolar) needle capable of delivering energy to tissue, for example, to cancerous or other diseased tissue (i.e., a tumor in a prostate). In these aspects, system 400 may be used to image one or more portions of a prostate (e.g., via ultrasound imaging with catheter 402, as discussed above) and also to apply energy (e.g., with needle 436) to treat the one or more portions of the prostate.

Insertion device 430 may be generally cylindrical, for example, including cylindrical lumens 432, 434. Alternatively, insertion device 430 may include a generally elliptical cross-section (e.g., lateral cross-section), and lumens 432, 434 may include one or more different cross-sectional shapes. Lumens 432, 434 may be generally the same size and shape. Alternatively, lumen 432 may be larger than lumen 434, or vice versa. Furthermore, insertion device 430 may be at least partially rigid and/or may include a size (e.g., a lateral cross-sectional size) of approximately 4 French to approximately 35 French, for example, approximately 12 French to approximately 35 French, or approximately 18 French to approximately 27 French (approximately 6 mm to approximately 9 mm), such that insertion device 430 may be delivered transurethrally.

Needle 436 may be a bipolar needle, for example, including a negative electrode portion 438 and a positive electrode portion 440. Electrode portions 438, 440 may be adjacent on a distal portion of needle 436, for example, proximal of distal end 436B. Although not shown, electrode portions 438, 440 may be coupled to respective conductive wires, for example, extending through needle 436 to a console or other capital equipment (e.g., including one or more power sources). Alternatively, needle 436 may be unipolar with multiple electrodes. For example, needle 436 may be monopolar with a ground pad (or return electrode). Moreover, in some aspects, distal end 436B may include a distal tip 436C, which may be sharpened or tapered, for example, to penetrate tissue, e.g., to pierce the wall of the urethra. Furthermore, in some aspects, a distal portion of needle 436 may include a pre-set shape, for example, such that the distal portion of needle 436 is torqueable, steerable, or otherwise deliverable to one or more target locations. In some aspects, the distal portion of needle 436 may be deflected by a pre-set or at least partially rigid portion, for example, at an angle between approximately 45 degrees and approximately 90 degrees. Alternatively or additionally, one or more portions of insertion device 430 may be steerable, for example, to direct needle 436 to one or more target locations.

As shown in FIG. 4B, components of system 400 may be introduced into a patient 410, for example, via a urethra 412. In some aspects, a gel (e.g., an ultrasonic gel) may be used to facilitate delivery of insertion device 430 into urethra 412, and/or one or more portions of insertion device 430 may include a hydrophilic coating, for example, to aid in insertion into and/or maneuvering within urethra 412. Insertion device 430 may be inserted with catheter 404 (e.g., surrounded by sheath 406) and positioned within one of lumens 432, 434, and needle 436 positioned within the other one of lumens 432, 434, such that catheter 404 and needle 436 are not extended distally of insertion device 430 (e.g., retracted within respective lumens 432, 434). Insertion device 430 may be advanced distally, for example, such that a distal portion of insertion device 430 is positioned adjacent to a prostate 414 and/or a bladder 416.

Furthermore, as shown in FIG. 4B, a distal portion (e.g., distal end 404B) of catheter 404 may be extended distally from insertion device 430, and/or insertion device 430 may be retracted proximally to expose the distal portion of catheter 404. In some aspects and as discussed above, catheter 404 may be retracted proximally or extended distally (e.g., in longitudinal direction A) and/or rotated (e.g., in direction B) in order to image one or more portions of prostate 414. In some aspects, the imaging with catheter 404 may identify suspicious tissue such as a tumor 442 or other targeted tissue. It is noted that, although not shown in FIG. 4B, catheter 404 may be positioned within sheath 406 (FIG. 4A) to help retain a fluid and/or protect catheter 404, as discussed above.

Once tumor 442 has been identified, needle 436 may be extended distally from insertion device 430 (or insertion device 430 proximally retracted), such that needle 436 may be delivered through the wall of urethra 412 and into prostate 414, for example, into one or more portions of tumor 442 and/or the targeted tissue. Moreover, as shown in FIG. 4A, needle 436 may include one or more markings 446 (e.g., echo markings) to help the operator track the position of needle 436. In some aspects, needle 436 may include a plurality of markings 446, for example, in different portions of needle 436 (e.g., on distal end 436B, negative electrode portion 438, positive electrode portion 440, etc.), and each of the different markings 446 may be a different size, shape, etc. In some aspects, markings 446 may also be on proximal end 436A for visual reference of distal end 436B depth of penetration distance and/or rotational orientation.

Once needle 436 is appropriately positioned, the operator may energize one or more portions of needle 436 (e.g., negative electrode portion 438, positive electrode portion 440, and/or a plurality thereof) to treat prostate 414 and/or tumor 442 at a treatment site 444 (e.g., an IRE treatment site). Needle 436 may be repositioned (e.g. extended distally, retracted proximally, inserted through different portions of the wall or urethra 412, etc.) as many times a needed to treat prostate 414 and/or tumor 442. Moreover, the treatment (e.g., one or more treatment sites 444) may be monitored via imaging with catheter 404.

In these aspects, system 400 may be used to perform various steps of method 300, and may also be used to deliver IRE therapy to one or more treatment sites 444. For example, system 400 may be delivered transurethrally (e.g., to a position adjacent to prostate 414 via urethra 412). Additionally, insertion device 430 may receive both catheter 404 (e.g., for IVUS imaging) and needle 436 within respective lumens 432, 434. Catheter 404 may be extended distally or retracted proximally (e.g., in longitudinal direction A) and/or rotated (e.g., in direction B) to help image or otherwise visualize one or more portions of prostate 414 (e.g., tumor 442). With the imaging or visualization, the operator may insert needle 436 to treat prostate 414 and/or tumor 442 at one or more treatment sites 444. Moreover, needle 436 and/or insertion device 430 may be repositioned (e.g., rotated, extended or retracted, deflected, etc.) in order for needle 436 to be inserted into prostate 414 and/or tumor 442. In some aspects, a first needle 436 may be positioned at treatment site 444 to delivery treatment or therapy. Then, first needle 436 may be proximally retracted or removed from insertion device 430, and a second needle 436 (e.g., of a different size, shape, insulation or electrode pattern, etc.) may be delivered and/or distally extended to deliver treatment or therapy, for example, at treatment site 444 or to another treatment site.

Needle(s) 436 may include different sizes and/or shapes (e.g., angular bends or deflections) depending on the size and/or position of tumor 442. Furthermore, the size and/or shape of negative electrode portion 438 and positive electrode portion 440 may vary between different needles 436, which may help to target different sizes and/or types of tumor 442. Additionally or alternatively, needle(s) 436 may include one or more insulated portions, for example, between or otherwise adjacent to (e.g., distal to or proximal of) negative electrode portion 438 and positive electrode portion 440. In some examples, needle 436 may be monopolar, e.g., with use of an external ground pad.

In these aspects, system 400, including catheter 404 and needle 436, may be delivered via urethra 412, which may be less invasive and/or less likely to lead to infection than other imaging, assessment, and/or treatment methods (e.g., transperineal or transrectal imaging/treatments). Moreover, catheter 404 may be positioned closer to prostate 414 when positioned in urethra 412, for example, compared to a catheter positioned in a rectum of patient 410. As such, catheter 404 may provide enhanced, clearer, more accurate, etc. visualization and imaging compared to transrectal imaging. Furthermore, transurethral imaging may help to guide the placement or delivery of needle 436 into prostate 414 and/or tumor 442, for example, to form treatment site(s) 444.

Needle 436 may be one or more of different needles, for example, including different sizes, angular bends, insulation patterns, electrode shapes and/or sizes, etc. As such, needle(s) 436 may help to allow for the operator to treat different shapes and/or sizes of tumors 442, for example, without over-treating or creating too large of a treatment site 444. Moreover, as mentioned, needle(s) 436 may be used to deliver IRE therapy. IRE therapy may include delivering a pulsed electrical field to tissue (e.g., prostate 414 and/or tumor 442) to at least partially ablate the tissue. Without being bound by theory, in some aspects, IRE may help to induce cell death, for example, while preserving surrounding structures. For example, IRE therapy may directly or indirectly cause or contribute to opening of one or more pores in a cell wall, which may help to result in apoptotic cell death. Without being bound by theory, it is believed that IRE therapy may, in some aspects, help to cause (directly or indirectly) the cells to release antigens, allowing the patient's own immune system to recognize and/or fight tumor 442 and the cancerous cells. In some aspects, other therapies (e.g., chemotherapy, radiation, and/or immunotherapy) may be used in conjunction with and/or after IRE therapy.

IRE therapy may help to spare or otherwise retain the extracellular matrix, for example, surrounding the cancerous cells, which may help to allow for quicker treatment and/or healing. IRE therapy may be used without applying heat to treatment site 444, avoiding damage to other portions of prostate 414 and/or other tissue surrounding treatment site 444. Furthermore, in some aspects, IRE therapy may help to provide an improved quality of life for patient 410 (e.g., relative to other cancer or tumor treatments like radiation therapies and/or chemotherapies), because IRE therapy may avoid or minimize negative effects on the nerves (e.g., nerve bundle) of prostate 414 and/or negative effects on the patient's vasculature in or surrounding prostate 414. Additionally, IRE therapy may reduce the need for other cancer, tumor, or hyperplasic tissue treatments, such as radiation therapies and/or chemotherapies. As such, IRE therapy may have a reduced likelihood of causing erectile dysfunction and/or incontinence compared to other prostate cancer therapies. It is noted, however, that the IRE therapy may be used in conjunction with one or more other cancer or tumor treatments (e.g., radiation therapies and/or chemotherapies).

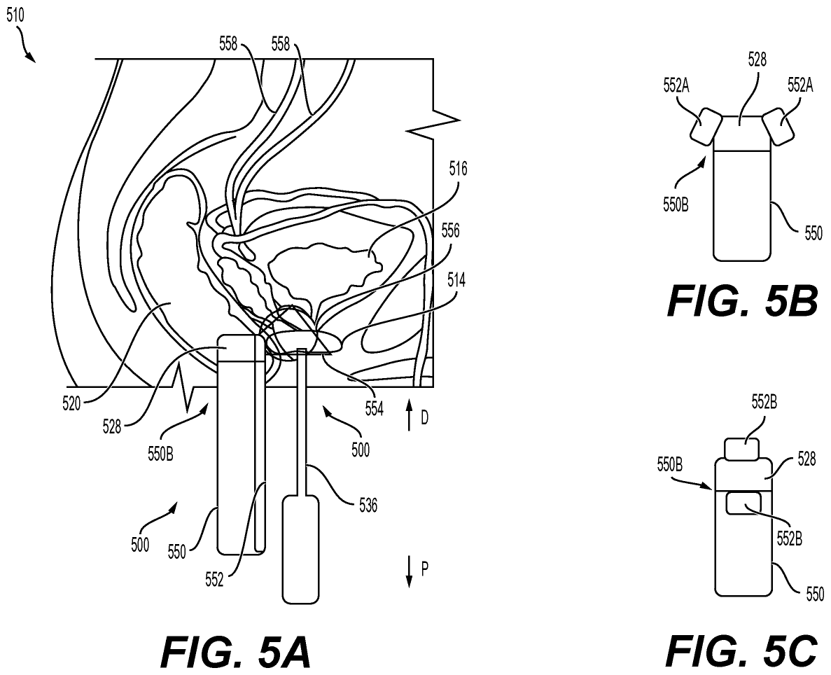

FIGS. 5A-5C illustrate another exemplary system 500 of this disclosure, which may be used for photoacoustic probing and/or ultrasonic motion biopsy. Furthermore, various components and aspects of system 500 may be used with one or more other therapies or diagnostics, for example, IRE therapy, cryotherapy, radio frequency therapy, etc.

As shown in FIG. 5A, system 500 includes an ultrasound probe 550 (e.g., a transrectal ultrasound probe) and a needle 536 (e.g., a linear motion biopsy needle or focal therapy probe needle). Ultrasound probe 550 includes one or more transducers 528, for example, on a distal end 550B of ultrasound probe. Moreover, system 500 includes an illumination device or a light source 552 (e.g., a laser light source), which may be part of an illumination device or included in a probe. In some aspects, as shown in FIG. 5A, light source 552 may be integrated into or otherwise part of ultrasound probe 550, for example, on a distal portion of ultrasound probe 550. System 500 may be used to perform one or more of transurethral, transrectal, and/or transperineal ultrasound imaging and photoacoustic imaging, for example, alternating between ultrasound imaging only, photoacoustic imaging only, and/or both ultrasound imaging and photoacoustic imaging. In these aspects, system 500 may allow for dual modality imaging or multimodal overlay imaging. Moreover, one or more aspects of system 500 may be used in conjunction with one or more other aspects of this disclosure, for example, insertion device 430, a steerable catheter, a steerable introducer, a rigid transperineal delivery device, etc.

For example, ultrasound probe 550 with light source 552 may be positioned in a rectum 520 of a patient 510. Additionally, needle 536 may be delivered to be adjacent to a prostate 514 of patient 510, for example, via a urethra or transperineally. Accordingly, an ultrasound field of view 554 for ultrasound probe 550 may at least partially overlap with an illumination area 556 from light source 552, and ultrasound field of view 554 and illumination area 556 may at least partially overlap with prostate 514. In these aspects, various aspects of system 500 may be used to illuminate and/or image various portions of patient 510 (e.g., the urethra, prostate 514, bladder 516, one or more ureters 558, etc.). Moreover, needle 536 may be used to biopsy or otherwise treat prostate 514 (e.g., including suspicious or diseased tissues such as a tumor), bladder 516, etc.

FIGS. 5B and 5C illustrate exemplary arrangements of light source(s) on a distal end of ultrasound probe 550. For example, as shown in FIG. 5B, two or more light sources 552A may be positioned on sides or corners of and/or extending from distal end 550B of ultrasound probe 550, for example, adjacent to the one or more transducers 528. Alternatively, as shown in FIG. 5C, two or more light sources 552B may be positioned along a longitudinal axis of ultrasound probe 550. For example, one or more light sources 552B may be positioned on distal end 550B of ultrasound probe 550 at a position on or distal to the one or more transducers 528. Furthermore, one or more light sources 552B may be positioned on distal end 550B of ultrasound probe 550 at a position proximal of the one or more transducers 528.

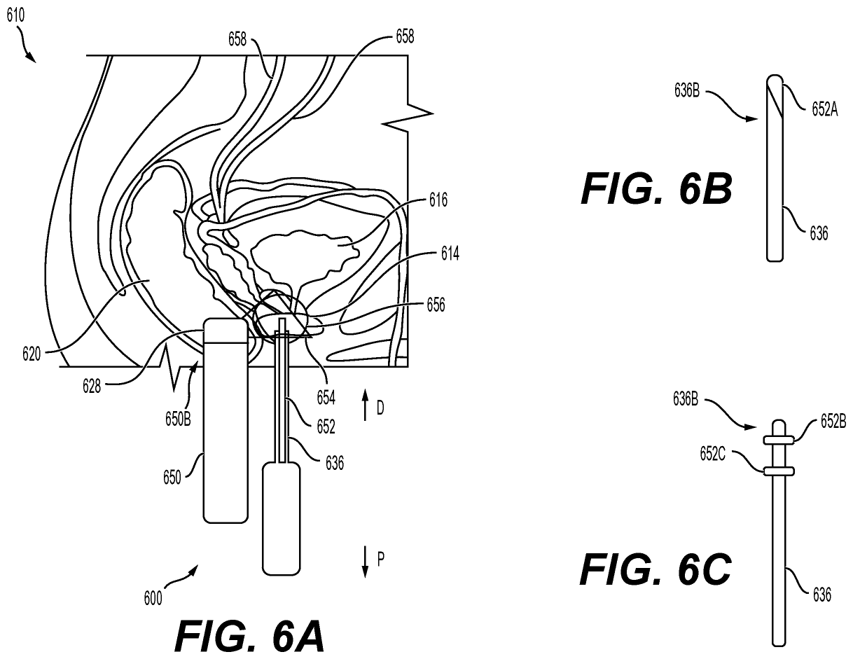

FIGS. 6A-6C illustrate another exemplary system 600 of this disclosure, which may be used for photoacoustic probing and/or ultrasonic motion biopsy. Furthermore, various components and aspects of system 600 may be used with one or more other therapies or diagnostics, for example, IRE therapy, cryotherapy, radio frequency therapy, etc.

As shown in FIG. 6A, system 600 includes an ultrasound probe 650 (e.g., a transrectal ultrasound probe) and a needle 636 (e.g., a linear motion biopsy needle). Ultrasound probe 650 includes one or more transducers 628, for example, on a distal end 650B of ultrasound probe 650. Moreover, system 600 includes an illumination device or a light source 652 (e.g., a laser light source). In some aspects, as shown in FIG. 6A, light source 652 may be integrated into, within, or otherwise part of needle 636, for example, on a distal portion of needle 636. System 600 may be used to perform transrectal ultrasound imaging and/or photoacoustic imaging, for example, alternating between ultrasound imaging only, photoacoustic imaging only, and/or both ultrasound imaging and photoacoustic imaging. In these aspects, system 600 may allow for dual modality imaging or multimodal overlay imaging.

For example, ultrasound probe 650 may be positioned in a rectum 620 of a patient 610. Additionally, needle 636 with light source 652 may be delivered to be adjacent to a prostate of patient 610, for example, via a urethra or transperineally. Accordingly, an ultrasound field of view 654 for ultrasound probe 650 may at least partially overlap with an illumination area 656 from light source 652 and ultrasound field of view 654 and illumination area 656 may at least partially overlap with prostate 614. In these aspects, various aspects of system 600 may be used to illuminate and/or image various portions of patient 610 (e.g., the urethra, prostate 614, bladder 616, one or more ureters 658, etc.). Moreover, needle 636 may be used to biopsy or otherwise treat prostate 614 (e.g., including a tumor), bladder 616, etc.

FIGS. 6B and 6C illustrate exemplary arrangements of light source(s) on a distal end 636B of needle 636. For example, as shown in FIG. 6B, one or more light sources 652A may be positioned internal to needle 636, for example within an internal lumen of needle 636. In some aspects, light source(s) 652A may be movable relative to needle 636. Alternatively, as shown in FIG. 6C, two or more light sources 652B, 652C may be positioned on needle 636, for example, on respective exterior portions of needle 636. For example, one or more first light sources 652B may be positioned on or adjacent to (e.g., spaced proximally from) distal end 636B of needle 636. Furthermore, one or more second light sources 652C may be positioned on needle 636 at a position proximal of the one or more first light sources 652B. Alternatively, light sources 652B, 652C may be internal to needle 636, for example, with needle 636 including respective openings or fenestrations for illumination from light sources 652B, 652C to illuminate respective portions of patient 610. In any of these aspects, positioning light source(s) 652, 652A, 652B, 652C on needle 636 may help to provide a greater depth of penetration for photoacoustic imaging.

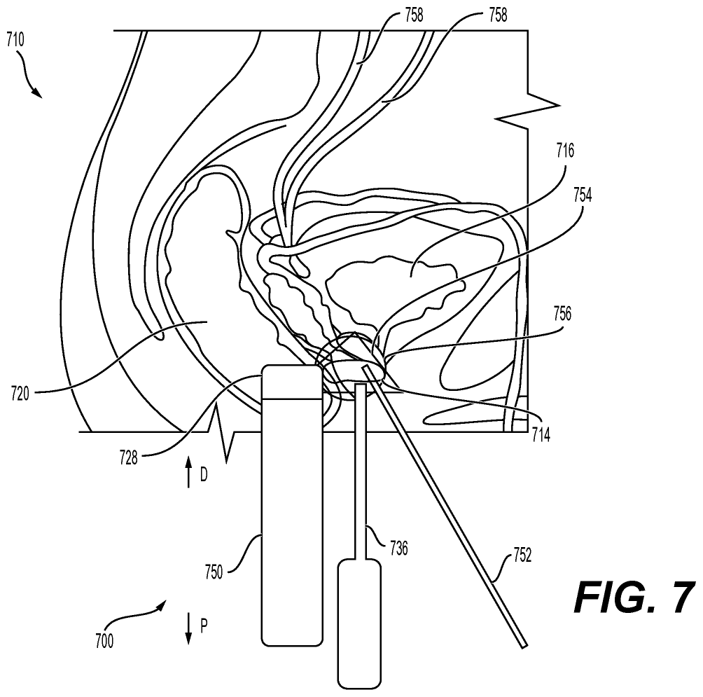

FIG. 7 illustrates another exemplary system 700 of this disclosure, which may be used for photoacoustic probing and/or ultrasonic motion biopsy. Furthermore, various components and aspects of system 700 may be used with one or more other therapies or diagnostics, for example, IRE therapy, cryotherapy, radio frequency therapy, etc.

As shown in FIG. 7, system 700 includes an ultrasound probe 750 (e.g., a transrectal ultrasound probe) and a needle 736 (e.g., a linear motion biopsy needle). Ultrasound probe 750 includes one or more transducers 728, as discussed above. Moreover, system 700 includes an illumination device or a light source 752 (e.g., a laser light source). In some aspects, as shown in FIG. 7, light source 752 may be separate from needle 736 and ultrasound probe 750. For example, light source 752 may be a needle (e.g., to be delivered transperineally), or light source 752 may be a flexible probe (e.g., inserted into the urethra). System 700 may be used to perform one or more of transrectal ultrasound imaging and photoacoustic imaging, for example, alternating between ultrasound imaging only, photoacoustic imaging only, and/or both ultrasound imaging and photoacoustic imaging. In these aspects, system 700 may allow for dual modality imaging or multimodal overlay imaging.

For example, ultrasound probe 750 may be positioned in a rectum 720 of a patient 710. Additionally, needle 736 may be delivered to be adjacent to a prostate 714 of patient 710, for example, transperineally. Furthermore, light source 752 may be delivered to a position adjacent the prostate 714, for example, via the urethra or transperineally. As discussed below, system 700 may include multiple light sources, and, for example, one light source may be delivered transurethrally and another light source may be delivered transperineally. An ultrasound field of view 754 for ultrasound probe 750 may at least partially overlap with an illumination area 756 from light source 752, and ultrasound field of view 754 and illumination area 756 may at least partially overlap with prostate 714. In these aspects, various aspects of system 700 may be used to illuminate and/or image various portions of patient 710 (e.g., the urethra, prostate 714, bladder 716, one or more ureters 758, etc.). Moreover, needle 736 may be used to biopsy or otherwise treat prostate 714 (e.g., including a tumor), bladder 716, etc. Furthermore, needle 736, ultrasound probe 750, and light source 752 may each be independently movable, for example, allowing for varied imaging angles and positions.

In some aspects, light source 752 may include multiple light sources, for example, on a single probe or multiple probes with light sources being delivered transurethrally. Multiple light sources may help to illuminate a larger illumination area 756. Furthermore, in some aspects, different light sources of the multiple light sources may operate at different frequencies or wavelengths, for example, to help (e.g., the operator, a processor or console) differentiate between different types of tissues.

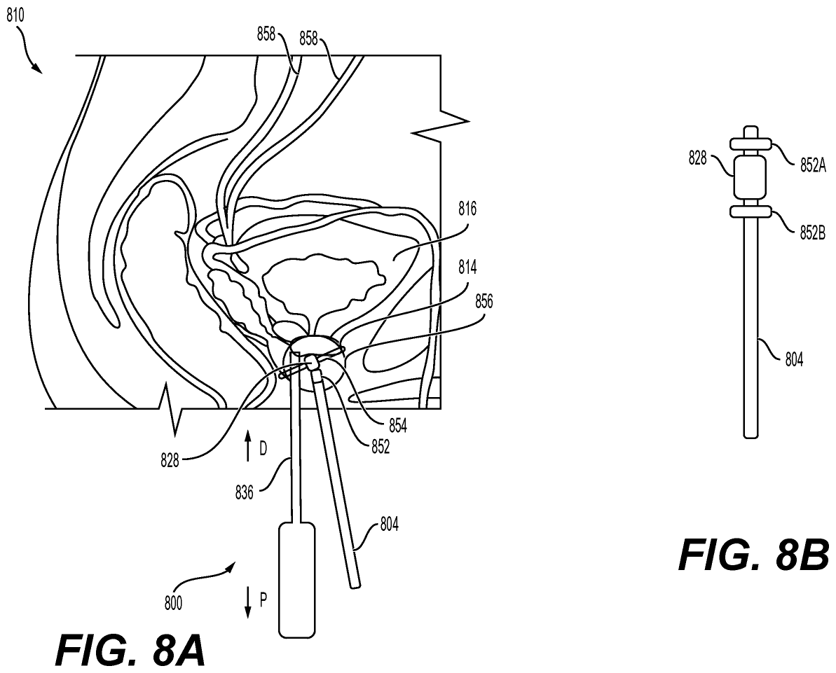

FIGS. 8A and 8B illustrate an additional exemplary system 800 of this disclosure, which may be used for photoacoustic probing and/or ultrasonic motion biopsy. Furthermore, various aspects of system 800 may be used with one or more other therapies or diagnostics, for example, IRE therapy, cryotherapy, radio frequency therapy, etc.

As shown in FIG. 8A, system 800 includes a catheter 804 and a needle 836 (e.g., a linear motion biopsy needle). Catheter 804 includes one or more light sources 852, for example, first and second light sources 852A, 852B, as shown in FIG. 8B (e.g., laser light sources). Additionally, catheter 804 includes a transducer 828 (e.g., an ultrasound transducer). Light source 852A may be positioned distal to transducer 828 on catheter 804, and light source 852B may be positioned proximal to transducer 828 on catheter 804.

In some aspects, as shown in FIG. 8A, catheter 804 and needle 836 are separate components, with catheter 804 forming an ultrasound probe for photoacoustic imaging (e.g., an IVUS catheter, as discussed above). For example, catheter 804 may be delivered transurethrally, as discussed above. Transducer 828 may form a radial probe, as discussed above, and may be activated to form an ultrasound field of view 854. Additionally, light sources 852A, 852B may be activated to illuminate one or more portions of patient 810, for example, prostate 814 and/or bladder 816, for example, to form an illumination area 856. In these aspects, catheter 804 may be delivered transurethrally and positioned adjacent to one or more portions of prostate 814, any may be activated to image prostate 814. Furthermore, light sources 852A, 852B may be activated to provide radial penetration of light, for example, into prostate 814, without requiring the delivery of additional devices. As discussed above, needle 836 may be delivered (e.g., transperineally) and utilized to biopsy or otherwise treat prostate 814 (e.g., including a tumor), bladder 816, etc. In these aspects, various aspects of system 800 may be used to illuminate and/or image various portions of patient 810 (e.g., the urethra, prostate 814, bladder 816, one or more ureters 858, etc.

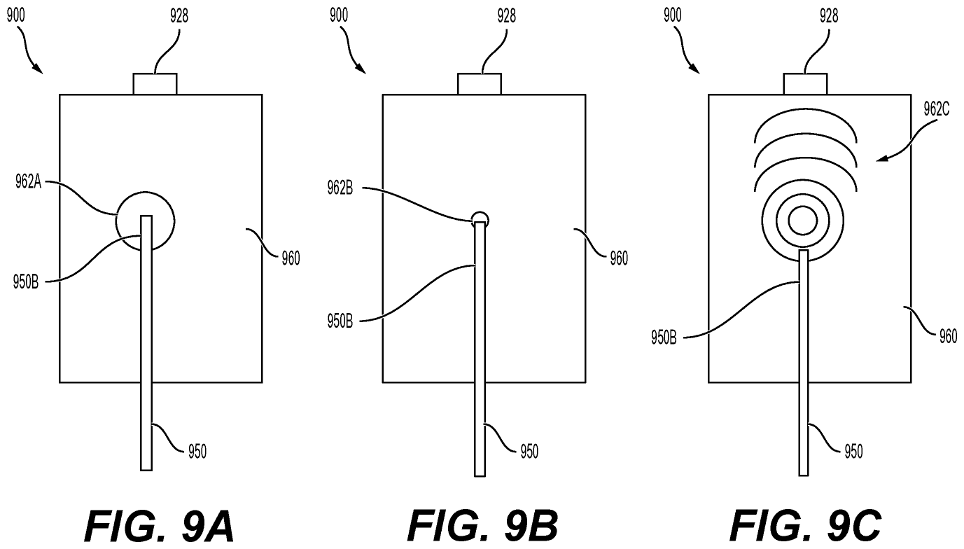

FIGS. 9A-9C illustrate an additional exemplary system 900 of this disclosure, which may be used for IRE signal-based acoustic probing. Furthermore, various aspects of system 900 may be used with one or more other therapies or diagnostics, for example, other types of IRE therapy, cryotherapy, radio frequency therapy, etc.

System 900 includes a probe 950, for example, an ultrasound probe or an IRE probe. System 900 also includes a receiver or a transducer 928, for example, spaced away from probe 950 by at least some tissue 960. For example, a distal end 950B of probe 950 may be positioned within or adjacent to tissue 960 (e.g., via a urethra) and transducer 928 may be positioned within or adjacent to tissue 960 (e.g., transrectally or transperineally). As shown in FIG. 9A, distal end 950B of probe 950 may be energized or otherwise activated to generate or otherwise create a bubble 962A (e.g., via ultrasound or IRE). As shown in FIG. 9B, bubble 962A may collapse to form a collapsed bubble 962B (e.g., at distal end 950B), for example, due to cavitation-type action. Then, as shown in FIG. 9C, collapsed bubble 962B may further collapse, burst, or otherwise generate energy in the form of acoustic waves 962C. Acoustic waves 962C created by the collapse of bubble 962A, 962B may be received by transducer 928.

In these aspects, system 900 may be used to deliver energy from probe 950 as input energy for acoustic imaging. The energy delivered by probe 950 may cause cavitation-type action or movement, for example, due to the pulsatile nature of the applied or delivered energy. The mechanical excitation may affect different types of tissues (e.g., healthy tissue compared to unhealthy or cancerous tissue) in different ways or to different levels. Accordingly, the mechanical excitation may produce an acoustic signal (e.g., as detected by transducer 928) readable by ultrasound at specific or different frequencies. In these aspects, the pulsatile exposure to high electric field may affect the tissue sufficiently to create a signal, for example, regardless of bubble formation or cavitation-type action or movement in tissue 960.

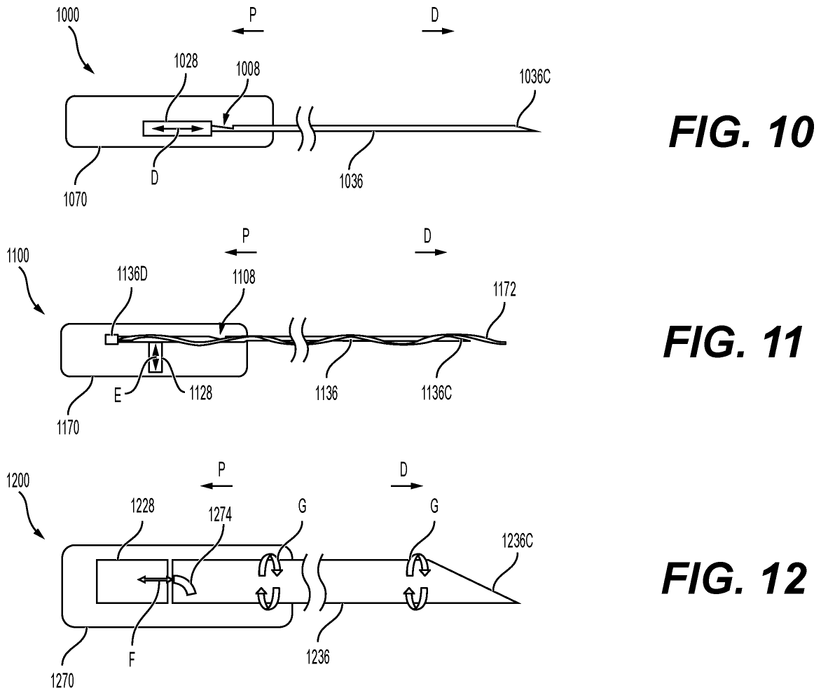

FIGS. 10-12 illustrate various additional needles 1036, 1136, and 1236, respectively, which may be incorporated in one or more of the systems discussed herein. For example, needles 1036, 1136, and 1236 may be actuated (e.g., with one or more piezoelectric transducers) to create, deliver, or otherwise apply ultrasonic energy for acoustic imaging and/or biopsy.

As shown in FIG. 10, an exemplary system 1000 includes needle 1036 and a handle 1070, which may be coupled to a proximal portion of needle 1036. Handle 1070 may include a transducer 1028, for example, a piezoelectric transducer, coupled to (e.g., directly or indirectly) to needle 1036. Moreover, needle 1036 may include an internal lumen, for example, including a proximal opening in a port 1008 (e.g., a stylet port). A distal end of needle 1036 may include a distal tip 1036C, which may be sharpened or tapered. Transducer 1028 may be oriented and/or positioned within handle 1070 and/or relative to needle 1036 such that the main or primary direction of expansion of the piezoelectric elements of transducer 1028 are collinear with the longitudinal axis of needle 1038, for example, in direction D. In these aspects, activation of transducer 1028 may deliver energy to needle 1036 to oscillate needle 1036 longitudinally, e.g., convey longitudinal movement in line with or parallel to direction D.

As shown in FIG. 11, an exemplary system 1100 includes needle 1136 and a handle 1170, which may be coupled to a proximal portion of needle 1136. Handle 1170 may include a transducer 1128, for example, a piezoelectric transducer, coupled to (e.g., directly or indirectly) to needle 1136. Moreover, needle 1136 may include an internal lumen, for example, including a proximal opening in a port 1108 (e.g., a stylet port). A distal end of needle 1136 may include a distal tip 1136C, which may be sharpened or tapered. Furthermore, a proximal portion or a proximal end 1136D of needle 1136 may be fixed to a portion of handle 1170, for example, to an internal portion of handle 1170. Transducer 1128 may be oriented and/or positioned within handle 1170 and/or relative to needle 1136 such that the main or primary direction of expansion of the piezoelectric elements of transducer 1128 are perpendicular to the longitudinal axis of needle 1138, for example, in direction E. In these aspects, activation of transducer 1128 may deliver energy to needle 1136 to oscillate needle 1136 harmonically, e.g., convey lateral movement approximated by wave 1172. Wave 1172 indicates a maximum deflection of needle 1136. In these aspects, a spacing between the fixed portion of needle 1136 (e.g., proximal end 1136D, and transducer 1128 may affect the harmonic length (e.g., between nodes of wave 1172), maximum deflection, or other features of wave 1172.

As shown in FIG. 12, an exemplary system 1200 includes needle 1236 and a handle 1270, which may be coupled to a proximal portion of needle 1236. Handle 1270 may include a transducer 1228, for example, a piezoelectric transducer, coupled to (e.g., directly or indirectly) to needle 1236. Although not shown, needle 1236 may include an internal lumen, for example, including a proximal opening in a port (e.g., a stylet port). A distal end of needle 1236 may include a distal tip 1236C, which may be sharpened or tapered. Transducer 1228 may be oriented and/or positioned within handle 1270 and/or relative to needle 1236 such that the main or primary direction of expansion of the piezoelectric elements of transducer 1228 (e.g., in direction F) cause rotational movement of needle 1238, for example, in direction G. Transducer 1228 may be coupled to needle 1236 via a screw 1274, for example, a lead screw, a power screw, a translation screw, or similar mechanism that transforms linear movement (e.g., of transducer 1228) to rotational movement (e.g., of needle 1236). In these aspects, activation of transducer 1228 may deliver energy to needle 1236 to rotate needle 1236, for example, an entire length of needle 1236 (e.g., convey rotational movement around a longitudinal axis in direction G).

Additionally, in some aspects, transducers 1028, 1128, 1228 may deliver energy to respective needles 1036, 1136, 1236 in order to cause mechanical motion (e.g., reciprocating longitudinal motion, oscillatory motion, or rotational motion) of approximately 0.03 inches or less, for example, approximately 0.01 inches or less. In various aspects of FIGS. 10-12, handles 1070, 1170, 1270 may be capital equipment and/or reusable, for example, with single-use or replaceable needles (e.g., needle shafts, distal tips, etc.). Alternatively, handles 1070, 1170, 1270, along with the needles, may be single-use devices. In these aspects, handles 1070, 1170, 1270, for example, including transducers 1028, 1128, 1228, may form self-contained devices to impart ultrasonic motion to one or more needles.

FIG. 13 illustrates an additional exemplary system 1300 of this disclosure, which may be used for IRE signal-based acoustic probing for imaging, assessing, and/or treating a prostate, bladder, or other tissue in a patient. Furthermore, various aspects of system 1300 may be used with one or more other therapies or diagnostics, for example, other types of IRE therapy, cryotherapy, radio frequency therapy, etc.

As shown, system 1300 includes a needle 1336 and an insertion device 1330. Insertion device 1330 includes a first shaft 1332 and a second shaft 1334, for example, with first shaft 1332 and second shaft 1334 coupled together. First shaft 1332 and second shaft 1334 may extend distally from a handle portion 1370. First shaft 1332 and second shaft 1334 may each include respective lumens 1332A and 1334A. One or more of first shaft 1332 and second shaft 1334 may form a working channel and/or may include a sharped or tapered distal end. In some aspects, lumen 1332A of first shaft 1332 may include an imaging unit 1378. Imaging unit 1378 may be built into first shaft 1332, or may removably positioned within lumen 1332A of first shaft 1332. Moreover, imaging unit 1378 may be a photoacoustic imaging and ultrasound imaging unit (e.g., forward or side looking), which may be activated for one or more of photoacoustic and/or ultrasound imaging, as discussed above. For example, imaging unit 1378 may comprise a catheter with one or more transducers, for example, movably positioned within a sheath, as discussed above.

Insertion device 1330 may also include a port 1308, for example, to receive a portion of needle 1336. In some aspects, port 1308 may be connected to lumen 1334A of second shaft 1334. A radial exterior portion of port 1308 may include a threading 1308A, for example, to form a female luer lock connection or otherwise help secure a portion of needle 1336 to port 1308. Insertion device 1330 may also include a connection portion 1380, for example, extending at an angle from handle portion 1370. Connection portion 1380 of insertion device 1330 may be coupled to a cable or umbilicus 1382, which may include or otherwise be coupled to an electronic connector 1384. Connector 1384 may be a photoacoustic imaging and ultrasound imaging connector, for example, to couple imaging unit 1378 to a photoacoustic imaging and/or ultrasound imaging unit (e.g., similar to console 102 of FIGS. 1A-1B and FIGS. 2A-2B). In these aspects, connection portion 1380, umbilicus 1382, and connector 1384 may couple imaging unit 1378 to one or more of a power source, a transducer, a processor, a display, or other equipment to activate and/or receive signals from imaging unit 1378.

Insertion device 1330 may be configured for transperineal insertion and/or delivery to the patient. For example, insertion device 1330 may be formed of a non-metallic material, and may form a dual lumen cannula. In some aspects, insertion device 1330 may be a single lumen cannula. As mentioned, distal ends of one or more of first shaft 1332 and/or second shaft 1334 may be sharpened or tapered. Moreover, one or more portions of insertion device 1330 (e.g., first shaft 1332 and/or second shaft 1334) may include one or more markings 1386 (e.g., echo and/or visual markings), for example, spaced longitudinally along one or more of first shaft 1332 and/or second shaft 1334. Markings 1386 may be identical, or may be different colors, sizes, shapes, etc. based on the position of respective markings 1386 on insertion device 1330. Additionally, markings 1386 may be regularly or evenly spaced on insertion device 1330, or may be irregularly spaced. In at least one aspect, markings 1386 on a distal portion of insertion device 1330 may be regularly spaced apart by a first distance, and markings 1386 on a proximal portion of insertion device 1330 may be irregularly spaced or regularly spaced apart by a second distance (e.g., smaller than the first distance). In any of these aspects, markings 1386 may help the operator to estimate the position and/or depth of penetration of insertion device 1330 (e.g., the distal ends of first shaft 1332 and/or second shaft 1334) within the patient.

Furthermore, needle 1336 includes a fitting 1388, for example, coupled to a proximal end 1336A of needle 1336. Moreover, needle 1336 includes a distal end 1336B, for example, including a distal tip 1336C, which may be sharpened or otherwise tapered. Fitting 1388 may include an internal threading, for example, to form a male luer lock connection that may be coupled to threading 1308A to secure needle 1336 to insertion device 1330 (e.g., with needle 1336 positioned within lumen 1334A of second shaft 1334). In some aspects, when needle 1336 is positioned within lumen 1334A of second shaft 1334, distal tip 1336C may extend distal of second shaft 1334 (e.g., shown in dashed lines in FIG. 13). Needle 1336 may be a rigid needle or trocar. In some aspects, needle 1336 may be metallic or at least partially metallic. In other aspects, needle 1336 may be non-metallic.

In some aspects, needle 1336 may be inserted within lumen 1334A of second shaft 1334 of insertion device 1330, or within the cannula (i.e., a working channel) if insertion device 1330 is a single lumen cannula. Needle 1336 may be delivered (e.g., inserted and/or steered) to an ablation or treatment site, for example, transperineally. In some aspects, external visualization may assist in the delivery of needle 1336 to the ablation or treatment site. After needle 1336 is used to deliver energy (e.g., for ablation), needle 1336 may be removed from lumen 1334A (or the working channel of a single lumen cannula). Then, a biopsy device, ablation catheter, imaging device, or other medical device may be inserted into lumen 1334A (or the working channel) and used to biopsy tissue at the treatment site, deliver ablation energy, perform imaging, or otherwise treat the treatment site. Additionally, if insertion device 1330 is a dual lumen catheter, one of the lumens (e.g., lumen 1332A of first shaft 1332 may be used for imaging, while another of the lumens (e.g., lumen 1334A of second shaft 1334) may be used for needle 1336 and/or one or more other medical devices. Furthermore, insertion device 1330 may include one or more additional lumens (e.g., in one or more of first shaft 1332 and second shaft 1334, or within an additional shaft). For example, lumen 1332A may be used for photoacoustic imaging with a light source and signal reception (e.g., photoacoustic imaging and/or ultrasound imaging with imaging unit 1378). Lumen 1334A may be used for needle 1336 and/or one or more other medical devices, and an additional lumen may be used for sending acoustic signals and receiving acoustic signals (or other imaging, assessment, or treatment).

As mentioned above, one or more aspects of system 1300 may be combined with IRE therapy, focal therapy, or one or more other therapies. Additionally, system 1300 may help to provide dual lumen access (e.g., dual lumen cannula access). Furthermore, various aspects of system 1300 may be used with one or more other aspects of this disclosure. In some aspects, insertion device 1330 and needle 1336 may be used to couple the needle, imaging devices, and/or light source for various therapies, for example, as discussed above with respect to FIGS. 4A-8B and/or to perform one or more steps of method 300 (FIG. 3).

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. A medical system, comprising:
a sheath configured to be positioned within a urethra of a patient, wherein a proximal portion of the sheath includes a port configured to receive one or more fluids; and
a catheter insertable into the sheath and configured to be movably positioned within the sheath in the urethra;
wherein a distal portion of the catheter includes one or more ultrasound transducers configured to perform ultrasound imaging of at least a portion of a prostate of the patient.

2. The medical system of claim 1, further comprising an insertion device with first and second lumens, one of the first lumen or second lumen being configured to receive the sheath or the catheter.

3. The medical system of claim 2, further comprising a needle, wherein at least a portion of the needle is configured to be movably positioned within the other of the first lumen or the second lumen of the insertion device.

4. The medical system of claim 3, wherein a distal portion of the needle includes a negative electrode portion and a positive electrode portion, the needle being configured to be energized for irreversible electroporation therapy.

5. The medical system of claim 4, wherein the distal portion of the needle includes one or more echo markings.

6. The medical system of claim 2, wherein a distal portion of one or both of the first lumen or the second lumen is sharpened or tapered.

7. The medical system of claim 3 wherein a proximal portion of the insertion device includes a female luer connection, and wherein a proximal portion of the needle includes a male luer connection.

8. The medical system of claim 2, wherein the insertion device further includes a connection portion, an umbilicus, and an electronic connector to operably couple insertion device and the catheter to a console.

9. The medical system of claim 3, wherein the needle is an ultrasonic needle coupled to a piezoelectric transducer.

10. The medical system of claim 9, wherein the piezoelectric transducer is configured to convey one or more of longitudinal movement, lateral movement, or rotational movement to the ultrasonic needle.

11. The medical system of claim 1, wherein the sheath is flexible, and wherein a distal end of the sheath is closed.

12. The medical system of claim 9, wherein the sheath is translucent to ultrasound waves.

13. The medical system of claim 1, further comprising a needle configured to be delivered to the prostate of the patient transperineally, the needle comprising at least one light source.

14. The medical system of claim 1, further comprising one or more light sources positioned on the distal portion of the catheter.

15. The medical system of claim 1, wherein the distal portion of the catheter includes two transducers.

16. A medical system, comprising:
an ultrasound probe insertable into a rectum of a patient, the ultrasound probe including a distal end comprising at least one transducer, wherein the at least one transducer, when energized, forms an ultrasound field of view that at least partially overlaps with a prostate of the patient;
an illumination device insertable into the rectum or a perineum of the patient, the illumination device including at least one light source that, when energized, forms an illumination area that at least partially overlaps with the prostate of the patient; and
a needle, wherein a distal end of the needle is configured to biopsy or treat the prostate of the patient.

17. The medical system of claim 16, wherein the illumination device is coupled to the ultrasound probe, and wherein the illumination device includes two light sources coupled to a distal end of the ultrasound probe, and wherein the two light sources are adjacent to the transducer.

18. The medical system of claim 16, wherein the illumination device includes the needle, the illumination device including two light sources coupled to a distal portion of the needle, wherein the ultrasound probe is configured to be delivered transrectally, and wherein the needle is configured to be delivered transperineally.

19. A method of treating a patient, comprising:
inserting a sheath and a catheter through a urethra of the patient, wherein the sheath is flexible and includes a port in a proximal portion of the sheath, the sheath having a closed distal end, and wherein a distal portion of the catheter includes at least one ultrasound transducer;
delivering a fluid to the sheath through the port such that the fluid is between the catheter and the sheath;
imaging a prostate of the patient by activating or energizing the at least one ultrasound transducer of the catheter and longitudinally or rotatably moving the catheter; and
removing the catheter and the sheath from the urethra.

20. The method of claim 19, wherein imaging the prostate includes obtaining multiple images by advancing the catheter distally, activating or energizing the at least one ultrasound transducer multiple times, and longitudinally or rotatably moving the catheter to ultrasonically image different portions of the prostate; and
wherein the method further comprises applying pressure to the prostate transrectally and performing one or more elastography procedures.
